(19) United States
(12) Patent Application Publication
(10) Pub. No.: US 2019/0265699 A1
(43) Pub. Date: Aug. 29, 2019

(54) CONCENTRATION DEGREE DETERMINATION DEVICE, CONCENTRATION DEGREE DETERMINATION METHOD, AND PROGRAM FOR DETERMINING CONCENTRATION DEGREE

(71) Applicant: OMRON Corporation, Kyoto-shi, KYOTO (JP)

(72) Inventors: Tomohiro YABUUCHI, Kyoto-shi (JP); Tomoyoshi AIZAWA, Kyoto-shi (JP); Madoka WATANABE, Kyoto-shi (JP)

(73) Assignee: OMRON Corporation, Kyoto-shi, KYOTO (JP)

(21) Appl. No.: 16/333,850

(22) PCT Filed: Oct. 25, 2017
(86) PCT No.: PCT/JP2017/038575

§ 371 Date: Mar. 15, 2019

(30) Foreign Application Priority Data
Mar. 14, 2017 (JP) 2017-048203

(51) Int. Cl. G05D 1/00; B60W 50/08; A61B 5/00; A61B 5/18

(52) U.S. Cl. CPC G05D 1/0061; B60W 50/082; A61B 5/0077; G05D 2201/0213; A61B 5/6893; B60W 2540/22; A61B 5/18

(57) ABSTRACT

A concentration degree determination device includes: an information acquisition unit configured to acquire a driving concentration degree of a driver of a vehicle; a reference setting unit configured to set a first reference or a second reference higher than the first reference for a manual driving mode that is switched from an automatic driving mode based on at least one of a first driving concentration degree and running information during execution of the automatic driving mode; a reference comparator configured to compare a second driving concentration degree during execution of the manual driving mode to the first or second reference; and an information output unit configured to output information according to a comparison result.

### TECHNICAL FIELD

The present invention relates to a concentration degree determination device that determines a concentration degree of a driver of a vehicle, a concentration degree determination method, and a program for determining the concentration degree.

### BACKGROUND ART

In recent years, in addition to a manual driving mode in which a vehicle is caused to run based on a driving operation of a driver, an automatic driving mode in which the vehicle is caused to run along a previously-set route regardless of the driving operation of the driver has been developed as an driving mode of a vehicle.

A technique of continuously running in the automatic driving section until timing when the user is awaken in the case that the user who drives the vehicle performing the automatic driving control is sleeping has also been developed (see Japanese Unexamined Patent Publication No. 2015-141053).

### SUMMARY OF THE INVENTION

Although drivers are required to secure driving safety regardless of the driving mode, driving concentration degree required for the driver varies according to a situation in which the driver is placed. The driving safety is maintained when the vehicle constantly monitors the driving concentration degree of the driver by a strict reference and issues a warning when the driving concentration degree falls below the reference. However, the vehicle frequently issues an alarm depending on the situation in which the driver is placed. In this case, driving comfort of the driver is impaired.

A desirable situation in which the driving concentration degree of the driver is monitored with a strict reference is assumed depending on the driving mode and the state of the driver.

The present invention has been made in view of the above circumstances, and an object of the present invention is to provide a concentration degree determination device, a concentration degree determination method, and a program for determining concentration degree for allowing both the driving safety and the driving comfort to be considered according to the driving mode and the state of the driver.

In order to solve the above problem, according to a first aspect of the present invention, a concentration degree determination device includes: an information acquisition unit configured to acquire a driving concentration degree of a driver of a vehicle; a reference setting unit configured to set a first reference or a second reference higher than the first reference for a manual driving mode that is switched from an automatic driving mode based on at least one of a first driving concentration degree and running information during execution of the automatic driving mode; a reference comparator configured to compare a second driving concentration degree during execution of the manual driving mode to the first or second reference; and an information output unit configured to output information according to a comparison result.

According to a second aspect of the present invention, in the concentration degree determination device of the first aspect, the first or second reference is set based on a sleep state of the driver included in an index of the first driving concentration degree.

According to a third aspect of the present invention, in the concentration degree determination device of the first or second aspect, the first or second reference is set based on a sleep time of the driver included in an index of the first driving concentration degree.

According to a fourth aspect of the present invention, in the concentration degree determination device of any one of the first to third aspects, the reference setting unit sets the first or second reference based on at least one of a running time, a running distance, and a road environment that are included in the running information.

According to a fifth aspect of the present invention, in the concentration degree determination device of any one of the first to fourth aspects, the reference setting unit sets the first or second reference based on at least one of the first driving concentration degree and the running information in a first period including a period immediately before the automatic driving mode is switched to the manual driving mode.

According to a sixth aspect of the present invention, in the concentration degree determination device of any one of the first to fifth aspects, the reference setting unit sets the first or second reference in a second period including a period immediately after the automatic driving mode is switched to the manual driving mode based on at least one of the first driving concentration degree and the running information.

According to a seventh aspect of the present invention, in the concentration degree determination device of the sixth aspect, the reference setting unit sets the first reference in the manual driving mode after the second period elapses.

According to an eighth aspect of the present invention, in the concentration degree determination device of the sixth aspect, the reference setting unit sets the first or second reference in the manual driving mode after the second period elapses based on the second driving concentration degree in the second period.

According to a ninth aspect of the present invention, a concentration degree determination method includes: an information acquisition step of acquiring a driving concentration degree of a driver of a vehicle; a reference setting step of setting a first reference or a second reference higher than the first reference for a manual driving mode that is switched from an automatic driving mode based on at least one of a first driving concentration degree and running information during execution of the automatic driving mode; a reference comparison step of comparing a second driving concentration degree during execution of the manual driving mode to the first or second reference; and an information output step of outputting information according to a comparison result.

According to a tenth aspect of the present invention, a program for determining concentration degree, the program causes a computer to execute processing of each unit included in the concentration degree determination device according to any one of the first to eighth aspects.

In the first aspect of the present invention, the concentration degree determination device can set the first reference or the second reference higher than the first reference for the manual driving mode that can be switched from the automatic driving mode based on at least one of the first driving concentration degree and the running information during the execution of the automatic driving mode. That is, the second driving concentration degree during the execution of the manual driving mode can be determined based on the first or second reference that is variably set based on the state during the execution of the automatic driving mode. The information such as a warning can be output according to the determination result. Thus, depending on the state during the execution of the automatic driving mode, the driving safety is maintained by outputting information such as a proper warning during the execution of the manual driving mode. Depending on the state during the execution of the automatic driving mode, the comfort is maintained by outputting information such as a proper warning during the execution of the manual driving mode. That is, in the first aspect, the concentration degree determination device can consider both the driving safety and the driving comfort according to the driving mode and the state of the driver.

In the second aspect of the present invention, the concentration degree determination device can set the first or second reference for the manual driving mode that can be switched from the automatic driving mode based on the sleep state during the execution of the automatic driving mode. That is, the second driving concentration degree during the execution of the manual driving mode can be determined based on the first or second reference that is variably set based on the sleep state during the execution of the automatic driving mode. For example, in the case where the driver is awake, the first reference is set and the determination reference is set to a normal level. In the case where the driver is asleep, sometimes the driver does not return to a wakefulness state, so that the second reference is set, and the determination reference is made stricter. Thus, depending on the sleep state during the execution of the automatic driving mode, the driving safety is maintained by outputting information such as a proper warning during the execution of the manual driving mode. Depending on the sleep state during the execution of the automatic driving mode, the comfort is maintained by outputting information such as a proper warning during the execution of the manual driving mode.

In the third aspect of the present invention, the concentration degree determination device can set the first or second reference for the manual driving mode that can be switched from the automatic driving mode based on the sleep time during the execution of the automatic driving mode. That is, the second driving concentration degree during the execution of the manual driving mode can be determined based on the first or second reference that is variably set based on the sleep time during the execution of the automatic driving mode. For example, in the case that the sleep time is shorter than the sleep reference time, the wakefulness state is assumed to be maintained, the first reference is set, and the determination reference is set to a normal level. In the case that the sleep time is greater than or equal to the sleep reference time, the driver is assumed not to return to the sufficient wakefulness state, the second reference is set, and the determination reference is made stricter. Conversely, in the case that the sleep time is greater than or equal to the sleep reference time, since the driver takes a sufficient rest, the first reference is set and the determination reference is set to the normal level. In the case that the sleep time is shorter than the sleep reference time, the driver is assumed to take an insufficient rest, the second reference is set, and the determination reference is made stricter. Thus, depending on the sleep time during the execution of the automatic driving mode, the driving safety is maintained by outputting information such as a proper warning during the execution of the manual driving mode. Depending on the sleep time during the execution of the automatic driving mode, the comfort is maintained by outputting information such as a proper warning during the execution of the manual driving mode.

In the fourth aspect of the present invention, the concentration degree determination device can set the first or second reference for the manual driving mode that can be switched from the automatic driving mode based on at least one of the running time, the running distance, and the road environment during the execution of the automatic driving mode. That is, the second driving concentration degree during the execution of the manual driving mode can be determined based on the first or second reference that is variably set based on the running state during the execution of the automatic driving mode. For example, in the case that the running time is shorter than the running reference time, a fatigue degree is assumed to be low, the first reference is set, and the determination reference is set to the normal level. In the case that the running time is greater than or equal to the running reference time, the fatigue degree is assumed to be high, the second reference is set, and the determination reference is made stricter. Alternatively, in the case that the running distance is shorter than the running reference distance, the fatigue degree is assumed to be low, the first reference is set, and the determination reference is set to the normal level. In the case that the running distance is greater than or equal to the running reference distance, the fatigue degree is assumed to be high, the second reference is set, and the determination reference is made stricter. Alternatively, in the case that the vehicle is running on the first road, the fatigue degree is assumed to be low, the first reference is set, and the determination reference is set to the normal level. In the case that the vehicle is running on the second road having the road environment in which the driver needs more concentration and attention on the driving of the vehicle as compared with the first road, the fatigue degree is assumed to be high, the second reference is set, and the determination reference is made stricter. Thus, depending on the running state during the execution of the automatic driving mode, the driving safety is maintained by outputting information such as a proper warning during the execution of the manual driving mode. Depending on the running state during the execution of the automatic driving mode, the comfort is maintained by outputting information such as a proper warning during the execution of the manual driving mode.

In the fifth aspect of the present invention, the concentration degree determination device can set the first or second reference based on at least one of the first driving concentration degree and the running information in the first period including the period immediately before the automatic driving mode is switched to the manual driving mode. The state immediately before the automatic driving mode is switched to the manual driving mode may have an influence on the state after the automatic driving mode is switched to the manual driving mode. Because the state immediately before the automatic driving mode is switched to the manual driving mode is considered, the driving safety is maintained by outputting information such as a proper warning during the execution of the manual driving mode. Depending on the state during the execution of the automatic driving mode, the comfort is maintained by outputting information such as a proper warning during the execution of the manual driving mode.

In the sixth aspect of the present invention, the concentration degree determination device can set the first or second reference in the second period including the period immediately after the automatic driving mode is switched to the manual driving mode based on at least one of the first driving concentration degree and the running information. The state immediately before the automatic driving mode is switched to the manual driving mode has a large influence on the state immediately after the automatic driving mode is switched to the manual driving mode. When the first or second reference is set immediately after the automatic driving mode is switched to the manual driving mode, the driving safety is maintained by outputting information such as a proper warning immediately after the execution of the manual driving mode. When the first or second reference is set immediately after the automatic driving mode is switched to the manual driving mode, the comfort is maintained by outputting information such as a proper warning immediately after the execution of the automatic driving mode.

In the seventh aspect of the present invention, the concentration degree determination device can set the first reference in the manual driving mode after the elapse of the second period. In the case that the first reference is set in the second period of the manual driving mode, the setting of the first reference is continued after the elapse of the second period. In the case that the second reference is set in the second period of the manual driving mode, the second reference set after the elapse of the second period is changed to the first reference. For example, even if the driver sleeps while executing the automatic driving mode, the driver is awaken when the second period elapses after the automatic driving mode is switched to the manual driving mode. When the first reference is set in the manual driving mode after the elapse of the second period, the driving safety is maintained by outputting information such as a proper warning during the execution of the manual driving mode. The comfort is maintained by outputting information such as a proper warning during the execution of the automatic driving mode.

In the eighth aspect of the present invention, the concentration degree determination device can set the first or second reference in the manual driving mode after the elapse of the second period based on the second driving concentration degree in the second period. That is, the determination reference of the driving concentration degree can be variably set in the third period after the second period based on the driving concentration degree in the second period. In the case that the immediately preceding driving concentration degree is high, the subsequent driving concentration degree can be set to the first reference. In the case that the immediately preceding driving concentration degree is low, the subsequent driving concentration degree can be set to the second reference. Consequently, the driving safety is maintained by outputting information such as a proper warning during the execution of the manual driving mode. The comfort is maintained by outputting information such as a proper warning during the execution of the automatic driving mode.

In the ninth aspect of the present invention, the concentration degree determination method can obtain the same effect as the first aspect. That is, the concentration degree determination method can consider both the driving safety and the driving comfort according to the driving mode and the state of the driver.

In the tenth aspect of the present invention, the program for determining the concentration degree can obtain the same effect as the first to eighth aspects. That is, the program for determining the concentration degree can consider both the driving safety and the driving comfort according to the driving mode and the state of the driver.

### BRIEF DESCRIPTION OF THE DRAWINGS

FIG. 1 is a view illustrating an overall configuration of a vehicle including a concentration degree determination device according to an embodiment of the present invention.

FIG. 2 is a block diagram illustrating a configuration of the concentration degree determination device of the embodiment of the present invention.

FIG. 3 is a block diagram illustrating a configuration of a state detector of the embodiment of the present invention.

FIG. 4 is a view illustrating a first example of a first period included in an execution period of an automatic driving mode.

FIG. 5 is a view illustrating a second example of the first period included in the execution period of the automatic driving mode.

FIG. 6 is a view illustrating a third example of the first period included in the execution period of the automatic driving mode.

FIG. 7 is a view illustrating another example of reset of a manual driving reference in a third period included in an execution period of a manual driving mode.

FIG. 8 is a flowchart illustrating a procedure of concentration degree determination by the concentration degree determination device in FIG. 2.

FIG. 9 is a flowchart illustrating a first example of a procedure of setting the manual driving reference of the concentration degree determination in FIG. 8.

FIG. 10 is a flowchart illustrating a second example of the procedure of setting the manual driving reference of the concentration degree determination in FIG. 8.

FIG. 11 is a flowchart illustrating a third example of the procedure of setting the manual driving reference of the concentration degree determination in FIG. 8.

### MODE FOR CARRYING OUT THE INVENTION

An embodiment of the present invention will be described below with reference to the drawings.

### Embodiment

### (Configuration)

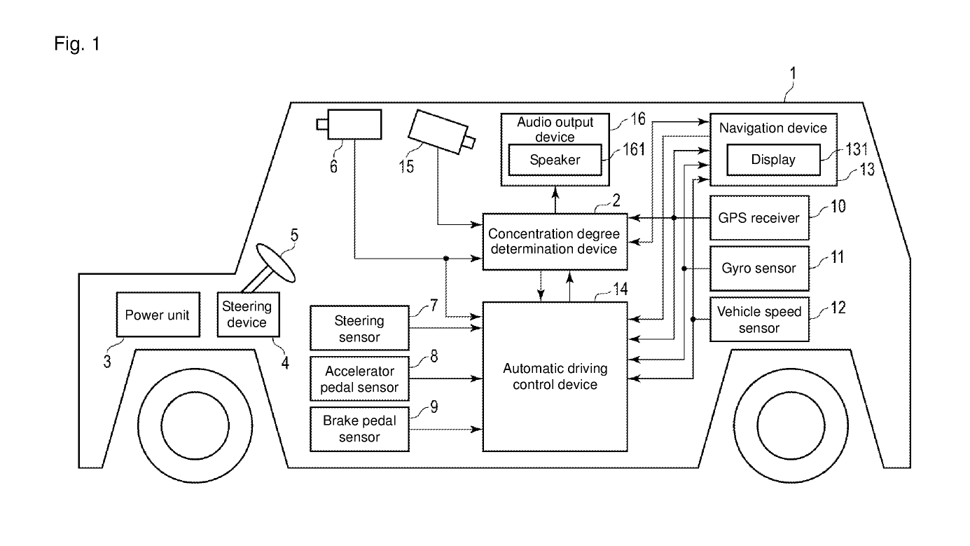

FIG. 1 is a view illustrating an overall configuration of a vehicle 1 including a concentration degree determination device 2 of an embodiment of the present invention. The concentration degree determination device 2 is mounted on the vehicle 1 such as a passenger car. A configuration of the concentration degree determination device 2 will be described later. For example, the vehicle 1 may be any one of a car, a bus, a truck, a train, and the like, or other vehicles on which a driver rides.

The vehicle 1 includes a power unit 3 including a power source and a speed change device and a steering device 4 equipped with a steering wheel 5 as basic equipment, and has a manual driving mode and an automatic driving mode as a driving mode. An engine, a motor, or both are used as the power source.

For example, the manual driving mode is a mode in which the vehicle 1 is mainly caused to run by a manual driving operation of the driver. For example, the manual driving mode includes an operating mode in which the vehicle 1 is caused to run based only on the driving operation of the driver and an operating mode in which driving operation support control to support the driving operation of the driver is performed while the driving operation of the driver is mainly performed.

For example, the driving operation support control assists steering torque such that steering of the driver becomes a proper steering amount based on a curvature of a curve when the vehicle 1 is running on the curve. The driving operation support control also includes control to support an accelerator operation (for example, an operation of an accelerator pedal) or a brake operation (for example, an operation of a brake pedal) of the driver, manual steering (manual driving of the steering), and manual speed adjustment (manual driving of speed adjustment). In the manual steering, the driver mainly operates the steering wheel 5 to steer the vehicle 1. In the manual speed adjustment, the speed of the vehicle 1 is adjusted mainly by accelerator operation or brake operation by the driver.

The driving operation support control does not include control to forcibly intervene the driving operation of the driver to cause the vehicle 1 to run automatically. That is, the manual driving mode includes control to reflect the driving operation of the driver in the running of the vehicle 1 within a previously-set allowable range, but does not include control to forcibly intervene the running of the vehicle 1 under a certain condition (for example, lane departure of the vehicle 1).

On the other hand, for example, the automatic driving mode is a mode in which a driving state in which the vehicle 1 is cause to run automatically along a running road of the vehicle 1 is performed. For example, the automatic driving mode includes a driving state in which the driver causes the vehicle 1 to run automatically toward a previously-set destination without performing the driving operation. In the automatic driving mode, the whole control of the vehicle 1 is not necessarily automatically performed. The automatic driving mode also includes a driving state in which the driving operation of the driver is reflected in the running of the vehicle 1 within a previously-set allowable range. That is, the automatic driving mode includes control to reflect the driving operation of the driver in the running of the vehicle 1 within the previously-set allowable range, and to forcibly intervene the running of the vehicle 1 under a certain condition.

The vehicle 1 further includes an external camera 6, a steering sensor 7, an accelerator pedal sensor 8, a brake pedal sensor 9, a GPS receiver 10, a gyro sensor 11, a vehicle speed sensor 12, a navigation device 13, an automatic driving control device 14, a driver camera 15, and an audio output device 16.

The external camera 6 is installed at any position of the vehicle 1 so as to capture an image of an outside of the vehicle 1. Although one external camera 6 is illustrated in FIG. 1, the vehicle 1 may include a plurality of external cameras that capture images in different directions. The external camera 6 continuously captures the image of a running environment in a vicinity of the vehicle 1. The external camera 6 is activated in response to start of driving of the vehicle 1, and continuously captures the image of the outside of the vehicle 1. The external camera 6 outputs the captured image (hereinafter, also referred to as “external image data”) to the concentration degree determination device 2 and the automatic driving control device 14.

The steering sensor 7 detects a steering angle. The steering sensor 7 outputs a detection result to the automatic driving control device 14. The accelerator pedal sensor 8 detects an operation amount of the accelerator pedal. The accelerator pedal sensor 8 outputs the detection result to the automatic driving control device 14. The brake pedal sensor 9 detects the operation amount of the brake pedal. The brake pedal sensor 9 outputs the detection result to the automatic driving control device 14. The GPS receiver 10 receives current position information about the vehicle 1. The GPS receiver 10 outputs the current position information to the concentration degree determination device 2, the navigation device 13, and the automatic driving control device 14. The gyro sensor 11 detects a behavior of the vehicle 1. The gyro sensor 11 outputs the detection result to the navigation device 13 and the automatic driving control device 14. The vehicle speed sensor 12 detects speed of the vehicle 1. The vehicle speed sensor 12 outputs the detection result to the navigation device 13 and the automatic driving control device 14.

The navigation device 13 is an example of a video display device including a display 131 that displays video. The navigation device 13 stores map information. The navigation device 13 extracts route information from a current position to a destination using information about the destination input by the driver or the like, the map information, and the current position information from the GPS receiver 10. The navigation device 13 displays the route information on the display 131. The navigation device 13 can also display information except for the route information on the display 131. The navigation device 13 outputs the route information to the concentration degree determination device 2 and the automatic driving control device 14.

The navigation device 13 extracts running information about the vehicle (for example, a running distance and a running time) using the current position information from the GPS receiver 10, the detection result from the gyro sensor 11, and the detection result from the vehicle speed sensor 12. The navigation device 13 displays running information about the vehicle on the display 131.

The navigation device 13 outputs the running information about the vehicle to the concentration degree determination device 2 and the automatic driving control device 14.

The route information may include not only information about a route from the current position to the destination but also information about a road environment from the current position to the destination. Some examples of the information about the road environment will be described. The information about the road environment may include information about a type of road through which the vehicle 1 passes from the current position to the destination. For example, the type of the road is divided into a road on which passage of a person is restricted and a road on which the passage of the person is not restricted. For example, the road on which the passage of the person is restricted is an expressway. The expressway can also be referred to as a superhighway. For example, the road on which the passage of the person is not restricted is an ordinary road.

The information about the road environment may include information about a speed limit of the road through which the vehicle 1 passes from the current position to the destination. The information about the road environment may include position information about an installed object on the road through which the vehicle 1 passes from the current position to the destination. For example, the installed object is a sign, or may be an object installed on the road. The information about the road environment may include the position information about the building in the vicinity of the road through which the vehicle 1 passes from the current position to the destination. The route information may include information except for the above example as information about the road environment.

Examples of the first road and the second road will be described. For example, the second road is a road having the road environment in which the driver needs more concentration and attention to drive the vehicle 1 as compared with the first road. The second road may be a road having the road environment different from that of the first road as described above, and is not limited to this example.

Some examples in which the second road is the road having the road environment in which the driver needs more concentration and attention to drive the vehicle 1 as compared with the first road will be described below, but the present invention is not limited to these examples. In one example, the first road is an expressway and the second road is an ordinary road. Usually people cannot jump out on the expressway. On the other hand, people can jump out on the ordinary roads. The expressway is a road having a straight line section longer than that of the ordinary road. For this reason, in the ordinary road, irrespective of the driving mode, the driver needs more concentration and attention to drive the vehicle 1 as compared with the expressway.

In another example, the first road is a road section including an intersection where a signal is installed among the ordinary roads, and the second road is a road section including an intersection where the signal is not installed among the ordinary roads. At the intersection where the signal is installed, there is a low possibility that people jump out. On the other hand, at the intersection where the signal is not installed, there is a high possibility that people jump out. For this reason, in the road section including the intersection where the signal is not installed, irrespective of the driving mode, the driver needs more concentration and attention to drive the vehicle 1 as compared with the road section including the intersection where the signal is installed.

The configuration of the automatic driving control device 14 will be described. The automatic driving control device 14 automatically controls the running of the vehicle 1 when the driving mode is the automatic driving mode. The automatic driving control device 14 automatically controls the running of the vehicle 1 by setting the driving mode to the automatic driving mode based on various pieces of information or various instructions, or stops the automatic control by switching the driving mode from the automatic driving mode to the manual driving mode. The manual driving can be performed when the driving mode is switched to the manual driving mode, namely, when the automatic control is stopped. For example, the navigation device 13 receives an instruction input of the automatic driving mode from the driver, and instructs the automatic driving control device 14 to operate in the automatic driving mode, and the automatic driving control device 14 sets the driving mode to the automatic driving mode to automatically control the running of the vehicle 1. The navigation device 13 receives setting information about an automatic driving section corresponding to stored map information, and issues an instruction of the automatic driving mode when it is detected that the vehicle 1 enters the automatic driving section based on the current position information from the GPS receiver 10, and the automatic driving control device 14 sets the driving mode to the automatic driving mode to automatically control the running of the vehicle 1. The automatic driving control device 14 notifies the concentration degree determination device 2 of the driving mode (the automatic driving mode or the manual driving mode).

The automatic driving control device 14 acquires the external image data from the external camera 6, the detection result from the steering sensor 7, the detection result from the accelerator pedal sensor 8, the detection result from the brake pedal sensor 9, the current position information from the GPS receiver 10, the detection result from the gyro sensor 11, the detection result from the vehicle speed sensor 12, and the route information from the navigation device 13. For example, the automatic driving control device 14 automatically controls the running of the vehicle 1 based on these pieces of information and traffic information acquired by road-to-vehicle communication.

For example, automatic control includes automatic steering (automatic driving of the steering) and automatic speed adjustment (automatic driving of the speed). The automatic steering is a driving state in which the steering device 4 is automatically controlled. The automatic steering includes a lane keeping assist system (LKAS). The LKAS automatically controls the steering device 4 such that the vehicle 1 does not deviate from a driving lane, for example, even if the driver does not perform the steering operation. Even during the performance of the LKAS, the steering operation of the driver may be reflected on the steering of the vehicle within a range (allowable range) in which the vehicle does not deviate from the driving lane. The automatic steering is not limited to the LKAS.

The automatic speed adjustment is a driving state in which the speed of the vehicle 1 is automatically controlled. The automatic speed adjustment includes adaptive cruise control (ACC). For example, the ACC performs constant speed control causing the vehicle 1 to run at a constant speed and at a previously-set speed in the case that a preceding vehicle does not exist in front of the vehicle 1, and the ACC performs follow-up control adjusting the vehicle speed of the vehicle 1 according to an inter-vehicle distance to the preceding vehicle in the case that the preceding vehicle exists in front of the vehicle 1. The automatic driving control device 14 decelerates the vehicle 1 in response to the brake operation (for example, the operation of the brake pedal) of the driver even while the ACC is currently performed. Even if the ACC is currently performed, the automatic driving control device 14 can accelerate the vehicle 1 in response to the accelerator operation (for example, the operation of the accelerator pedal) of the driver up to a previously-set maximum permissible speed (for example, a legally-defined maximum speed on the road on which the vehicle is running). The automatic speed adjustment is not limited to not only the ACC but also cruise control (CC: constant speed control).

The configuration of the driver camera 15 will be described. For example, the driver camera 15 is installed at a position, such as on a dashboard, which faces a front of the driver. The driver camera 15 is an example of a sensor that monitors the driver. The driver camera 15 is activated in response to the start of the driving of the vehicle 1, and continuously captures an image of a predetermined range including a face of the driver. The driver camera 15 outputs the captured image (hereinafter, referred to as driver image data) to the concentration degree determination device 2. The driver image data is an example of monitoring data used to detect the state of the driver. For example, the state of the driver includes at least one of indices such as front gazing of a driver, sleepiness, sleep, looking aside, putting-on and taking-off of clothes, a telephone operation, leaning against a window side or an armrest, driving interference by a passenger or a pet, onset of a disease, back-facing, lying face down, eating and drinking, smoking, dizziness, an abnormal behavior, a car navigation or audio operation, putting-on and taking-off of glasses or sunglasses, image capturing, and visual recognition. The visual recognition means an index how much the driver visually recognizes the object. The state of the driver may include an index except for the indices exemplified here.

The audio output device 16 includes a speaker 161. The audio output device 16 outputs various pieces of information by sound.

The configuration of the concentration degree determination device 2 will be described. The concentration degree determination device 2 estimates a driving concentration degree of the driver based on the state of the driver and determines whether the driver is suitable for driving the vehicle 1. The driving concentration degree is a degree to which the driver is suitable for the driving of the vehicle 1. With increasing driving concentration degree, the driver becomes more suitable for the driving of the vehicle 1. On the other hand, with decreasing driving concentration degree, the driver becomes under a condition that the driver is not suitable for the driving of the vehicle 1. The driving concentration degree can also be said to be a driving risk degree. With increasing driving concentration degree, the driving risk degree is lowered to improve the safety. Conversely, with decreasing driving concentration degree, the driving risk degree is increased to lower the safety.

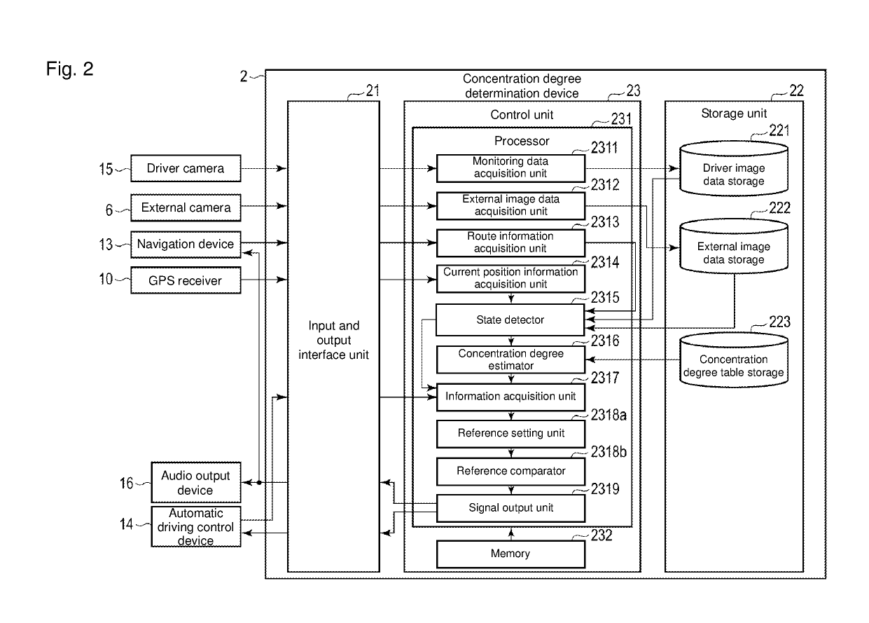

FIG. 2 is a block diagram illustrating the configuration of the concentration degree determination device 2 as an example. The concentration degree determination device 2 includes an input and output interface unit 21, a storage unit 22, and a control unit 23.

The input and output interface unit 21 connects each of the external camera 6, the GPS receiver 10, the navigation device 13, the automatic driving control device 14, the driver camera 15, and the audio output device 16 to the control unit 23.

The configuration of the storage unit 22 will be described. The storage unit 22 is a nonvolatile memory, such as a solid state drive (SSD) and a hard disk drive (HDD), in which writing and reading can be performed at any time. The storage unit 22 includes a driver image data storage 221, an external image data storage 222, and a concentration degree table storage 223.

The driver image data storage 221 stores the driver image data that is acquired from the driver camera 15 by the control unit 23. The external image data storage 222 stores the external image data that is acquired from the external camera 6 by the control unit 23.

The concentration degree table storage 223 stores a concentration degree table that is used by the control unit 23 to estimate the driving concentration degree. For each index, the concentration degree table correlates the state of the driver while dividing the state of the driver into a plurality of degrees according to the driving concentration degree. For example, the plurality of degrees are divided into three levels of a level 1, a level 2, and a level 3. However, the present invention is not limited to this configuration. At this point, an example in which the driving concentration degree is set lower as the level number increases will be described. However, the present invention is not limited to this example. The driving concentration degree may be set higher as the level number increases.

Information managed in the concentration degree table will be described by taking looking aside as an example. The concentration degree table correlates the state of the driver with the level 1, the level 2, and the level 3 with respect to the looking aside as an index. For example, the level 1 is correlated with the state of the driver who looks at a direction inclined at an angle within the range greater than or equal to 0 degrees and less than a first angle with respect to a traveling direction of the vehicle 1. That is, the level 1 is a state in which the driver is not looking aside but has the high driving concentration degree. For example, the level 2 is correlated with the state of the driver who looks at the direction inclined at an angle within a range greater than or equal to the first angle and less than a second angle with respect to the traveling direction of the vehicle 1. That is, the level 2 is a state, in which the driver is slightly looking aside and the driving concentration degree is lower than that of the level 1. For example, the level 3 is correlated with the state of the driver who looks at the direction inclined at an angle within the range greater than or equal to the second angle with respect to the traveling direction of the vehicle 1. That is, the level 3 is a state, in which the driver is looking aside and the driving concentration degree is lower than that of the level 2. In this case, the information managed in the concentration degree table is described by taking the looking aside as an example. The same holds true for other indices.

The configuration of the control unit 23 will be described. The control unit 23 includes a processor 231 and a memory 232. For example, the processor 231 is a central processing unit (CPU) constituting a computer. The configuration of each unit of the processor 231 will be described later. Although one processor 231 is illustrated in FIG. 2, the control unit 23 may include at least one processor. The memory 232 is provided with a program causing the processor 231 to function as processing of each unit of the processor 231. The program can also be referred to as an instruction to operate the processor 231. The program is stored in the storage unit 22, and read from the storage unit 22 to the memory 232. The program of the memory 232 is read by the processor 231. One embodiment may be implemented by the program.

The configuration of each unit of the processor 231 will be described. The processor 231 includes a monitoring data acquisition unit 2311, an external image data acquisition unit 2312, a route information acquisition unit 2313, a current position information acquisition unit 2314, a state detector 2315, a concentration degree estimator 2316, an information acquisition unit 2317, a reference setting unit 2318a, a reference comparator 2318b, and a signal output unit 2319. Each unit may be distributed to at least one processor.

The monitoring data acquisition unit 2311 acquires the driver image data from the driver camera 15 through the input and output interface unit 21. The monitoring data acquisition unit 2311 stores the driver image data in the driver image data storage 221. The external image data acquisition unit 2312 acquires the external image data from the external camera 6 through the input and output interface unit 21. The external image data acquisition unit 2312 stores the external image data in the external image data storage 222.

The route information acquisition unit 2313 acquires the route information from the navigation device 13 through the input and output interface unit 21. The route information acquisition unit 2313 outputs the route information to the state detector 2315. The current position information acquisition unit 2314 acquires the current position information from the GPS receiver 10 through the input and output interface unit 21. The current position information acquisition unit 2314 outputs the current position information to the state detector 2315.

The state detector 2315 detects the state of the driver from the driver image data stored in the driver image data storage 221. In addition to the driver image data, the state detector 2315 may detect the visual recognition as the state of the driver using at least one of the external image data, the route information, and the current position information. A detection example of the state of the driver by the state detector 2315 will be described later. The state detector 2315 may acquire the driver image data from the monitoring data acquisition unit 2311 with no use of the driver image data storage 221. In this case, the storage unit 22 may not include the driver image data storage 221. The state detector 2315 outputs the state of the driver to the concentration degree estimator 2316.

For example, the state detector 2315 detects the road environment included in the running information about the vehicle using the route information and the current position information. The state detector 2315 may detect the road environment included in the running information about the vehicle using the information obtained by road-to-vehicle communication. In addition to the route information and the current position information, the state detector 2315 may detect the road environment using the external image data. The state detector 2315 may determine the road environment of the road on which the vehicle 1 is running based on pieces of information except for the route information, the current position information, and the external image data. The state detector 2315 outputs the road environment to the information acquisition unit 2317.

The concentration degree estimator 2316 estimates the driving concentration degree of the driver based on the state of the driver detected by the state detector 2315. The state of the driver is detected from the driver image data as described above, so that the concentration degree estimator 2316 can also estimate the driving concentration degree of the driver from the driver image data. The concentration degree estimator 2316 estimates the driving concentration degree corresponding to each of at least one index included in the state of the driver. For example, the concentration degree estimator 2316 estimates the driving concentration degree using the sleepiness as an index, and also estimates the driving concentration degree with the looking aside as an index. For example, the concentration degree estimator 2316 may estimate one driving concentration degree by comprehensively determining the plurality of indices included in the state of the driver.

In one example, the concentration degree estimator 2316 can estimate the driving concentration degree using a numerical value such as a ratio. The numerical value estimated by the concentration degree estimator 2316 may increase with increasing driving concentration degree, or decrease with increasing driving concentration degree.

In another example, the concentration degree estimator 2316 can refer to the concentration degree table stored in the concentration degree table storage 223, and estimate the level of the driving concentration degree corresponding to the state of the driver from the plurality of levels. In the case that the concentration degree estimator 2316 estimates the driving concentration degree using the numerical value, the storage unit 22 may not include the concentration degree table storage 223.

The driving concentration degree may be estimated by the concentration degree estimator 2316 using an artificial intelligence (AI) function such as machine learning and deep learning. In this case, for example, the concentration degree estimator 2316 can accurately estimate the state of the driver by utilizing the past estimation result in the estimation of the current driving concentration degree. The concentration degree estimator 2316 outputs the driving concentration degree to the information acquisition unit 2317.

The information acquisition unit 2317 acquires information about the driving mode (automatic driving mode or manual driving mode) from the automatic driving control device 14 through the input and output interface unit 21. The information acquisition unit 2317 acquires the driving concentration degree from the concentration degree estimator 2316. The information acquisition unit 2317 acquires a running distance, running time, and the like as running information output from the navigation device 13 through the input and output interface unit 21. The information acquisition unit 2317 acquires the road environment and the like as the running information output from the state detector 2315.

The reference comparator 2318b compares the driving concentration degree estimated by the concentration degree estimator 2316 to the reference. The reference comparator 2318b compares the driving concentration degree to an automatic driving reference in the case that the driving mode of the vehicle 1 is the automatic driving mode. On the other hand, the reference comparator 2318b compares the driving concentration degree to a first manual driving reference or a second manual driving reference in the case that the driving mode is the manual driving mode.

For example, the first manual driving reference is a reference higher than the automatic driving reference, and the second manual driving reference is a reference higher than the first manual driving reference. In other words, the first manual driving reference is a stricter reference for the driving concentration degree than the automatic driving reference, and the second manual driving reference can be said to be a stricter reference for the driving concentration degree than the first manual driving reference. The automatic driving reference, the first manual driving reference, and the second manual driving reference may be arbitrarily changeable. In the case that the concentration degree estimator 2316 estimates the driving concentration degree with respect to each of the plurality of indices, the reference comparator 2318b may compare the driving concentration degree with respect to each of the plurality of indices to the reference. The reference comparator 2318b outputs the comparison result to the signal output unit 2319. The above reference can also be defined as a risk degree determination level (threshold for driving improper determination).

The comparison between the driving concentration degree estimated by the reference comparator 2318b using the numerical value and the reference will be described as an example. The case that the numerical value estimated by the concentration degree estimator 2316 increases with increasing driving concentration degree will be described below. The reference is set to a numerical value A. When the numerical value estimated by the concentration degree estimator 2316 is smaller than the numerical value A that is the reference, the reference comparator 2318b determines that the driving concentration degree estimated by the concentration degree estimator 2316 is lower than the reference.

The case that the numerical value estimated by the concentration degree estimator 2316 decreases with increasing driving concentration degree will be described below. When the numerical value estimated by the concentration degree estimator 2316 is larger than the numerical value A that is the reference, the reference comparator 2318b determines that the driving concentration degree estimated by the concentration degree estimator 2316 is lower than the reference.

The comparison between the driving concentration degree estimated at the level by the reference comparator 2318b and the reference will be described as another example. The reference is set to a level B extracted from a plurality of levels. In the case that the driving concentration degree lower than the level B that is the reference is assigned to the level estimated by the concentration degree estimator 2316, the reference comparator 2318b determines that the driving concentration degree estimated by the concentration degree estimator 2316 is lower than the reference.

An example in which the concentration degree table correlates the state of the driver with each index while dividing the state of the driver into three levels of the level 1, the level 2, and the level 3 will specifically be described. For example, the reference is set to the level 1. The reference comparator 2318b determines that the level 2 or level 3 estimated by the concentration degree estimator 2316 is lower than the level 1 that is the reference. On the other hand, the reference comparator 2318b determines that the level 1 estimated by the concentration degree estimator 2316 is not lower than the level 1 that is the reference.

The signal output unit 2319 outputs a signal to each unit through the input and output interface unit 21. Examples of some signals output from the signal output unit 2319 will be described below.

Based on the comparison result from the reference comparator 2318b, the signal output unit 2319 determines whether to output an instruction signal instructing the performance of the support to the driver to a support providing device. For example, the signal output unit 2319 outputs an instruction signal in the case that the driving concentration degree estimated by the concentration degree estimator 2316 is lower than the reference. Upon receiving the instruction signal from the signal output unit 2319, the support providing device performs predetermined support to the driver. For example, the support providing device is the navigation device 13 or the audio output device 16.

Based on the instruction signal, the navigation device 13 displays a warning giving attention to the driver on the display 131 as an image or video. Based on the instruction signal, the audio output device 16 outputs the warning giving the attention to the driver from the speaker 161 as a sound. The warning is not limited to a specific output mode, as long as the warning is a content that gives attention to the driver, for example, that the driving concentration degree is low or that it is necessary to concentrate on the driving. The driver can recognize that the driver is not in the state suitable for the driving of the vehicle 1 by the warning, and concentrate on the driving of the vehicle 1 again. The signal output unit 2319 may output the instruction signal to the support providing device except for the navigation device 13 and the audio output device 16. The signal output unit 2319 may output the instruction signal to the support providing device that gives an external stimulus such as vibration to the driver. The support to the driver is not limited as long as the support is the output content acting on the driver based on the driving concentration degree, and includes various kinds of support to encourage improvement of the driving concentration degree in addition to the warning, call for attention, and information provision.

The signal output unit 2319 can output a warning signal in the case that at least one driving concentration degree out of a plurality of driving concentration degrees estimated from a plurality of indices is lower than the reference. The signal output unit 2319 may output the warning signal when at least a predetermined number of driving concentration degrees out of the plurality of driving concentration degrees estimated from the plurality of indices is lower than the reference.

The signal output unit 2319 may output a switching signal switching the driving mode to the automatic driving control device 14. For example, in the case that the output condition of the warning signal is satisfied in the manual driving mode, the signal output unit 2319 may output the warning signal while outputting the switching signal switching the driving mode from the manual driving mode to the automatic driving mode to the automatic driving control device 14.

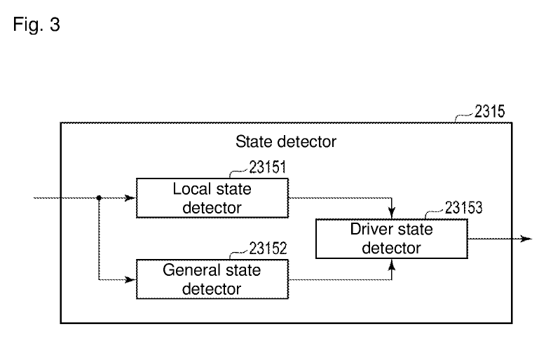

A detection example of the state of the driver using the driver image data by the state detector 2315 will be described below. A method of detecting the state of the driver is not limited to the example described below. FIG. 3 is a block diagram illustrating the configuration of the state detector 2315. For example, the state detector 2315 includes a local state detector 23151, a general state detector 23152, and a driver state detector 23153.

The local state detector 23151 detects the state of at least one of organs included in a face of the driver in the driver image data. Examples of the organs included in the face include eyes, a mouth, a nose, and ears. In the case that the local state detector 23151 detects the state of the eyes, for example, the local state detector 23151 detects a degree of opening and closing of the eyes of the driver, a direction of a line of sight, an orientation of the face, and the like. The local state detector 23151 outputs the detection result (hereinafter, also referred to as local information) to the driver state detector 23153.

The general state detector 23152 detects at least one state out of the general states of the driver in the driver image data. Examples of the general states include the operation and the attitude of the driver. The general state detector 23152 outputs the detection result (hereinafter, also referred to as general information) to the driver state detector 23153.

The driver state detector 23153 detects the state of the driver using the local information from the local state detector 23151 and the general information from the general state detector 23152. In this way, for example, by combining the local information and the general information, the state detector 2315 can detect various states of the driver.

Some detection examples of the visual recognition by the state detector 2315 will be described below. The state detector 2315 can detect the visual recognition using the monitoring data and the position information about the object.

By way of example, the state detector 2315 can detect the following visual recognition using the external image data in addition to the driver image data. The state detector 2315 extracts the object from the external image data in order to detect the visual recognition. For example, the object is an installed object such as a sign or a building, but the object is not particularly limited as long as the object has a possibility of being visually recognized by the driver. The state detector 2315 detects the line of sight and the orientation of the face of driver from the driver image data captured at substantially the same timing as the timing of capturing the external image data from which the object is extracted. The line of sight and the orientation of the face of the driver are detected by the local state detector 23151 as described above. The state detector 2315 detects the visual recognition using at least one of the line of sight and the orientation of the face of the driver and the position information about the object. The visual recognition increases as the line of sight and the orientation of the face of the driver are directed toward the object.

As another example, using the route information and the current position information in addition to the driver image data, the state detector 2315 can detect the visual recognition as follows. The state detector 2315 refers to the route information and the current position information, and extracts the object located in the vicinity of the vehicle 1. For example, as described above, the object is an installed object such as a sign or a building, but the object is not particularly limited as long as the object has a possibility of being visually recognized by the driver. The state detector 2315 detects the line of sight and the orientation of the face of the driver from the driver image data captured at substantially the same timing as the timing at which the vehicle 1 passes in the vicinity of the object. The state detector 2315 detects the visual recognition using at least one of the line of sight and the orientation of the face of the driver and the position information about the object.

As another example, the state detector 2315 may obtain the position of the object and the timing at which the vehicle 1 passes in the vicinity of the object by road-to-vehicle communication. In this case, the state detector 2315 detects the line of sight and the orientation of the face of the driver from the driver image data captured at substantially the same timing as the timing at which the vehicle 1 passes in the vicinity of the object. The state detector 2315 detects the visual recognition using at least one of the line of sight and the orientation of the face of the driver and the position information about the object.

As another example, the state detector 2315 may use a message displayed on the display 131 of the navigation device 13 as the object. In this case, the state detector 2315 detects the line of sight and the orientation of the face of the driver from driver image data captured at substantially the same timing as the timing of displaying the message on the display 131. The state detector 2315 detects the visual recognition using at least one of the line of sight and the orientation of the face of the driver and the position information about the object.

Using at least the monitoring data and the position information about the object as described above, the state detector 2315 can properly detect the state of the driver with the visual recognition as the index. The state detector 2315 may use the object located in the vicinity of the front, rear, left, or right side of the vehicle 1. Preferably the state detector 2315 uses the object located in the vicinity of the left or right side of the vehicle 1 as compared with the front side of the vehicle 1. The line of sight and the face of the driver do not move so much when the object is located on the front side of the vehicle 1. On the other hand, when the object is located in the vicinity of the left or right side of the vehicle 1, the line of sight and the face of the driver move to the left or right side. Consequently, the state detector 2315 can properly detect the visual recognition.

(Definition of Each Period)

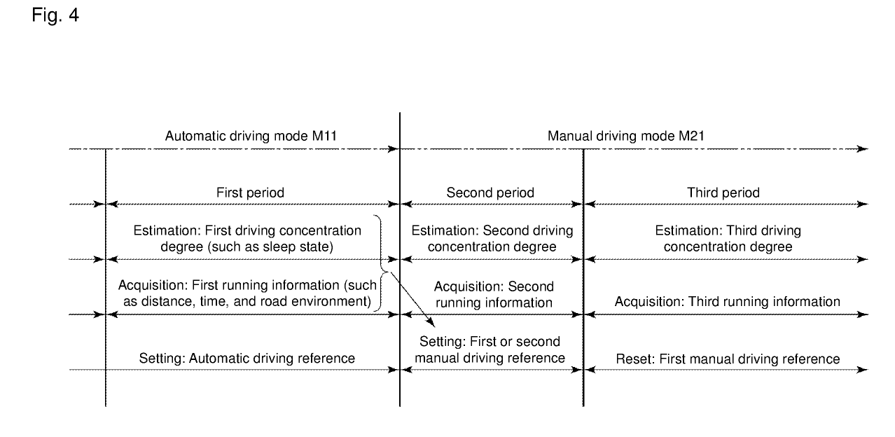

FIG. 4 is a view illustrating a first example of the first period included in the execution period of an automatic driving mode M11. As illustrated in FIG. 4, for example, the first period included in the execution period of the automatic driving mode M11 is a predetermined period including a period immediately before the switching to a manual driving mode M21. The predetermined period may be set to any time. For example, the predetermined period may be set to 90 minutes, 60 minutes, or 30 minutes. The driving concentration degree estimated (acquired) according to the first period is defined as a first driving concentration degree, and the running information acquired according to the first period is defined as first running information. The automatic driving reference is set corresponding to the execution period of the automatic driving mode M11. In the first example, all the periods of the automatic driving mode M11 are not set to the first period, but a part of the periods of the automatic driving mode M11 is set to the first period. For example, in the case that the execution period of the automatic driving mode M11 is extremely long, a restricted first period can be set, and the determination can be made based on the first driving concentration degree corresponding to the restricted first period.

The second period included in the execution period of the manual driving mode M21 is a predetermined period including a period immediately after the switching from the automatic driving mode M11 to the manual driving mode M21. The second period may be 30 minutes, 20 minutes, or 10 minutes. The driving concentration degree estimated (acquired) according to the second period is defined as a second driving concentration degree, and the running information acquired according to the second period is defined as second running information. The first or second manual driving reference is set according to the second period based on at least one of the first driving concentration degree and the first running information. How the first or second manual driving reference is selected will be described later in detail.

The third period included in the execution period of the manual driving mode M21 is a period after the second period elapses. The driving concentration degree estimated (acquired) according to the third period is defined as a third driving concentration degree, and the running information acquired according to the third period is defined as third running information. The first manual driving reference is set according to the third period.

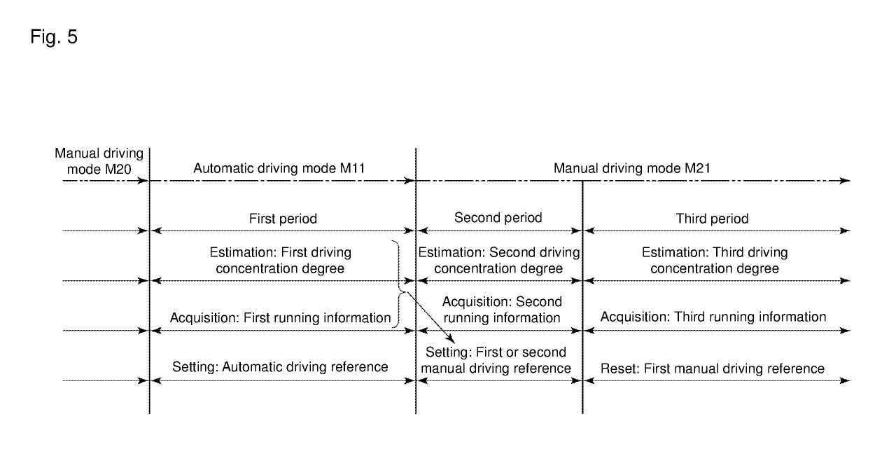

FIG. 5 is a view illustrating a second example of the first period included in the execution period of the automatic driving mode M11. As illustrated in FIG. 5, for example, the first period is a period including a period immediately before the switching to the manual driving mode M21, and is a period corresponding to the execution period of the automatic driving mode M11. The driving concentration degree estimated (acquired) according to the first period is defined as a first driving concentration degree, and the running information acquired according to the first period is defined as first running information. In the second example, all the continuous execution periods of the automatic driving mode M11 are substantially set to the first period. Consequently, the determination can be made based on the state of the driver corresponding to the whole period from the start to the end of the automatic driving mode M11, namely, the first driving concentration degree.

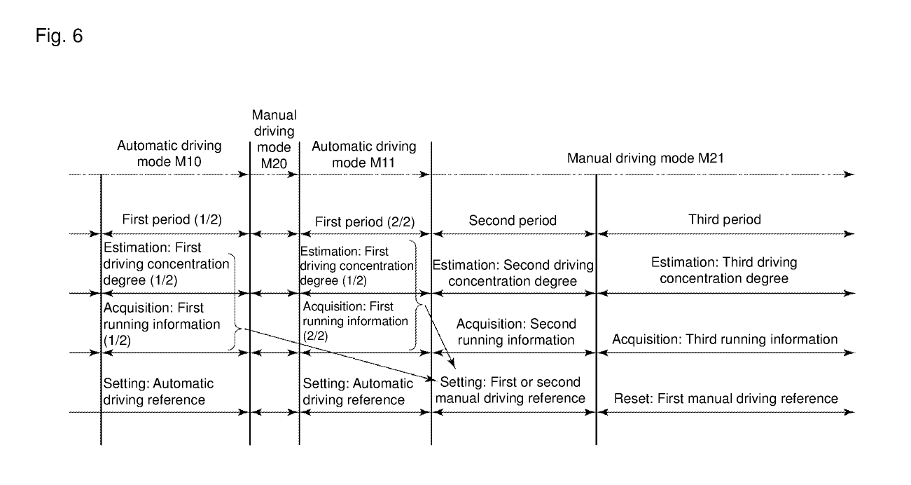

FIG. 6 is a view illustrating a third example of the first period included in the execution period of the automatic driving mode M11. As illustrated in FIG. 6, for example, the first period (1/2 and 2/2) may be discontinuous. The first period includes the execution period of a plurality of automatic driving modes (for example, automatic driving modes M10 and M11), and is a period including a period immediately before the switching to the manual driving mode M21. The discontinuous period is the execution period of the manual driving mode M20, for example, the discontinuous period is limited to 3 minutes or less. As a result, even if the manual driving mode M20 of a relatively short period is interrupted between the automatic driving modes M10 and M11, the driving concentration degree in the interrupted manual driving mode has a small influence on the driving concentration degrees in the automatic driving modes M10 and M11. For this reason, a plurality of discontinuous automatic driving modes as described above may be set to the first period.

The driving concentration degree estimated (acquired) according to the first period (1/2 and 2/2) is defined as a first driving concentration degree (1/2 and 2/2), and the running information acquired according to the first period is defined as first running information (1/2 and 2/2).

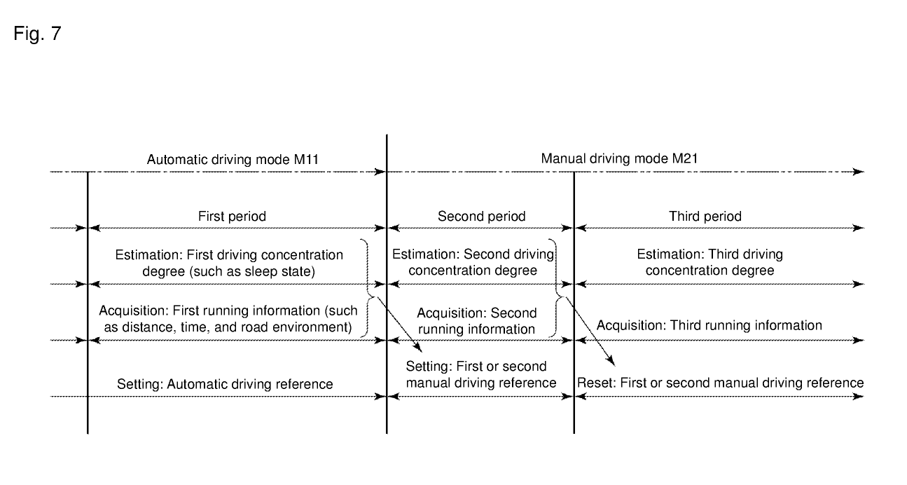

FIG. 7 is a view illustrating another example of reset of a manual driving reference in the third period included in the execution period of the manual driving mode M21. As illustrated in FIG. 7, the first or second manual driving reference is set according to the third period of the execution period of the manual driving mode M21 based on at least one of the second driving concentration degree and the second running information in the second period of the execution period of the manual driving mode M21. How the first or second manual driving reference is selected will be described later in detail.

(Operation)

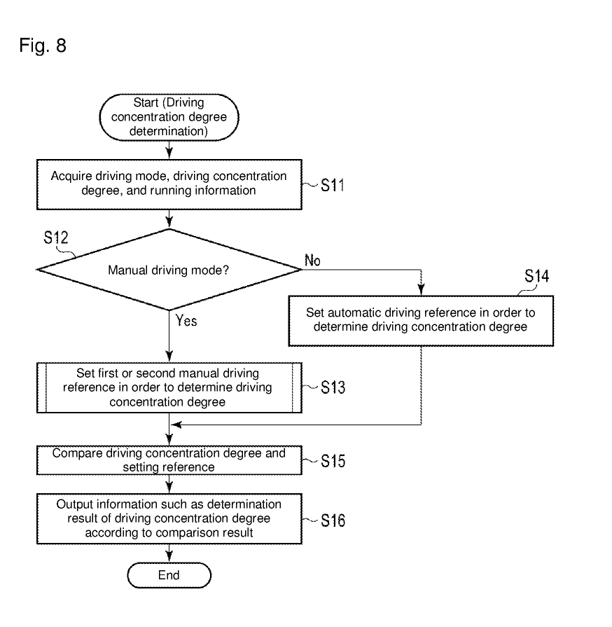

The operation of the concentration degree determination device 2 configured as described above will be described below. FIG. 8 is a flowchart illustrating an example of driving concentration degree determination processing by the concentration degree determination device 2. A flow of the estimation of the driving concentration degree will be described. The monitoring data acquisition unit 2311 acquires monitoring data from a sensor that monitors the driver of the vehicle 1. For example, the monitoring data acquisition unit 2311 acquires the driver image data from the driver camera 15 through the input and output interface unit 21. An interval at which the monitoring data acquisition unit 2311 acquires the monitoring data may be equal to or shorter than an interval at which the state detector 2315 detects the state of the driver.

Subsequently, the state detector 2315 detects the state of the driver from the monitoring data. For example, the state detector 2315 detects the state of the driver from the driver image data. For example, the state detector 2315 can detect the state of the driver at predetermined constant intervals. The state detector 2315 may detect the state of the driver at different intervals even if the state of the driver is detected at the same interval between the automatic driving mode and the manual driving mode. The state detector 2315 may detect the state of the driver in any timing.

Subsequently, the concentration degree estimator 2316 estimates the driving concentration degree of the driver from the monitoring data. For example, the concentration degree estimator 2316 estimates the driving concentration degree based on the state of the driver detected from the driver image data by the state detector 2315.

The information acquisition unit 2317 acquires the information about the driving mode (the automatic driving mode or the manual driving mode), the driving concentration degree, and the running information (such as the running distance, the running time, and the road environment) (step S11). The reference setting unit 2318a sets the reference determining the driving concentration degree based on the driving mode, the driving concentration degree, and the running information (steps S12 to S14).

For example, when the driving mode is the automatic driving mode (NO in step S12), the reference setting unit 2318a sets the automatic driving reference determining the driving concentration degree (step S14). At least one driving concentration degree among the plurality of driving concentration degrees estimated from the plurality of indices is set to the determination object, and the automatic driving reference corresponding to each driving concentration degree that becomes the object is set.

When the driving mode is switched from the automatic driving mode to the manual driving mode (YES in step S12), the reference setting unit 2318a sets the first or second manual driving reference in order to determine the second driving concentration degree during the execution of the manual driving mode that can be switched from the automatic driving mode based on at least one of the first driving concentration degree during the execution of the automatic driving mode, the state of the driver, and the first running information (step S13).

In the case of using the driving concentration degree, at least one driving concentration degree among the plurality of driving concentration degrees estimated from the plurality of indices is set to the determination object, and the first or second manual driving reference corresponding to each driving concentration degree that becomes the object is set. For example, the reference setting unit 2318a determines that the running in the automatic driving mode is relatively safe and sets the first manual driving reference in order to determine the second driving concentration degree in the manual driving mode when the first driving concentration degree in the automatic driving mode is greater than or equal to the reference, and the reference setting unit 2318a determines that the running in the automatic driving mode is dangerous and sets the second manual driving reference in order to determine the second driving concentration degree in the manual driving mode when the first driving concentration degree in the automatic driving mode is lower than the reference. The second manual driving reference is set and the second manual driving reference may be changed to the first manual driving reference after a predetermined period elapses. The second driving concentration degree during the execution of the manual driving mode is monitored, and the second manual driving reference may be changed to the first manual driving reference in the case that at least a given time elapses while the second driving concentration degree is greater than or equal to the second manual driving reference.

The setting of the first or second manual driving reference by the reference setting unit 2318a will be described later in detail.

The reference comparator 2318b compares the driving concentration degree to the reference to which the driving concentration degree is set, namely, one of the automatic driving reference, the first manual driving reference, and the second manual driving reference (step S15). The signal output unit 2319 outputs information such as the determination result of the driving concentration degree according to the comparison result. For example, in the case that the driving concentration degree is lower than the reference, the signal output unit 2319 outputs the warning signal. In the case that the driving concentration degree is lower than the reference, the signal output unit 2319 may output a deceleration control signal or a stop control signal. Based on the deceleration control signal or the stop control signal, the automatic driving control device 14 controls the brake operation to decelerate or stop the vehicle. In the case that the driving concentration degree is greater than or equal to the reference, the signal output unit 2319 may output driving concentration degree information. For example, the driving concentration degree information includes image information, and the navigation device 13 displays an image illustrating the driving concentration degree as a percentage based on the image information.

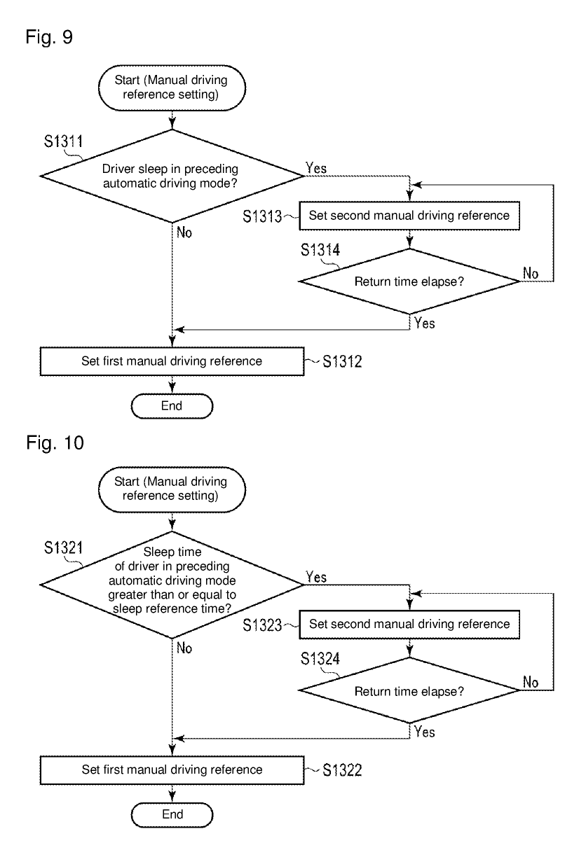

FIG. 9 is a flowchart illustrating a first reference setting example of the manual driving mode by the reference setting unit 2318a. The reference setting unit 2318a sets the first or second manual driving reference in the second period (see FIG. 4) included in the execution period of the manual driving mode according to a sleep state of the driver in the first period (see FIG. 4) included in the execution period of the automatic driving mode.

For example, the state detector 2315 detects the sleep state or a sleepy state (hereinafter, referred to as a sleepiness state) of the driver from the driver image data. The state detector 2315 may detect a state in which the eyes are closed consecutively for more than or equal to a sleep determination time (for example, 10 seconds) as the sleep state from an opening and closing degree of driver's eyes. The state detector 2315 may detect the state in which the eyes are continuously closed for time longer than a blinking time (for example, 1.5 seconds) from the opening and closing degree of the driver's eyes as the sleepiness state. The state detector 2315 may detect a state that corresponds to neither the sleep state nor the sleepiness state as a wakefulness state.

The concentration degree estimator 2316 estimates the driving concentration degree corresponding to each of one or more indices included in the state of the driver detected by the state detector 2315. For example, the concentration degree estimator 2316 estimates the driving concentration degree corresponding to the index of the sleep state. For example, the driving concentration degree corresponding to the index of the sleep state is low in the case where the driver is asleep, and high in the case where the driver is awake. Alternatively, the driving concentration degree corresponding to the index of the sleep state may be information about the case where the driver is asleep or the case where the driver is awake. The reference setting unit 2318a determines whether the driver is asleep, from the driving concentration degree corresponding to the index of the sleep state. In the case where the driver is awake (NO in step S1311), the reference setting unit 2318a sets the first manual driving reference (step S1312). In the case where the driver is asleep (YES in step S1311), the reference setting unit 2318a sets the second manual driving reference (step S1313). The reference setting unit 2318a maintains the setting of the second manual driving reference (step S1313) until a return time (second period) elapses (NO in step S1314), and the reference setting unit 2318a changes the second manual driving reference to the first manual driving reference (step S1312) when the return time elapses (YES in step S1314). That is, the reference setting unit 2318a sets the first manual driving reference in the third period after the elapse of the second period. For example, the return time corresponds to the second period, and may be set to time derived from the statistical data or the like that is required until a person in the sleep state shifts to the wakefulness state after waking.

The reference setting unit 2318a may set the first or second manual driving reference in the second period included in the execution period of the manual driving mode according to the sleepiness state of the driver in the first period included in the execution period of the automatic driving mode.

The first or second manual driving reference may be set using the sleep state or the sleepiness state detected by the state detector 2315 instead of the driving concentration degree corresponding to the index of the sleep state or the sleepiness state estimated by the concentration degree estimator 2316.

FIG. 10 is a flowchart illustrating a second reference setting example of the manual driving mode by the reference setting unit 2318a. The reference setting unit 2318a sets the first or second manual driving reference in the second period included in the execution period of the manual driving mode according to a sleep time of the driver in the first period included in the execution period of the automatic driving mode.

For example, the state detector 2315 detects the time of the sleep state. The state detector 2315 may detect the time of the sleepiness state. The state detector 2315 may detect the time of the wakefulness state.

For example, the concentration degree estimator 2316 estimates the driving concentration degree corresponding to the index of the sleep time. For example, the driving concentration degree corresponding to the index of the sleep time becomes lower with increasing sleep time. Alternatively, the driving concentration degree corresponding to the index of the sleep time is increased when a certain length of the sleep time (for example, 10 minutes or more and 20 minutes or less) is acquired, and the driving concentration degree corresponding to the index of the sleep time may be lowered as the sleep time deviates from the certain length of the sleep time. Alternatively, the driving concentration degree corresponding to the index of the sleep time may be information indicating the sleep time. The reference setting unit 2318a determines the sleep time of the driver from the driving concentration degree corresponding to the sleep state. When the sleep time is shorter than a sleep reference time (NO in step S1321), the reference setting unit 2318a sets the first manual driving reference (step S1322). When the sleep time is greater than or equal to the sleep reference time (YES in step S1321), the reference setting unit 2318a sets the second manual driving reference (step S1323). The reference setting unit 2318a maintains the setting of the second manual driving reference (step S1323) until the return time (second period) elapses (NO in step S1324), and the reference setting unit 2318a changes the second manual driving reference to the first manual driving reference (step S1322) when the return time elapses (YES in step S1324). That is, the reference setting unit 2318a sets the first manual driving reference in the third period after the elapse of the second period. In the case that it is known that the concentration degree of the driver is increased after the relatively short sleep time while the concentration degree of the driver is lowered after the relatively long sleep time, the first or the second manual driving reference is effectively set based on the sleep time. The length of the sleep reference time may be changed in consideration of a nap effect.

The first or second manual driving reference may be set using the sleep state or the sleepiness state detected by the state detector 2315 instead of the driving concentration degree corresponding to the index of the sleep state or the sleepiness state estimated by the concentration degree estimator 2316.

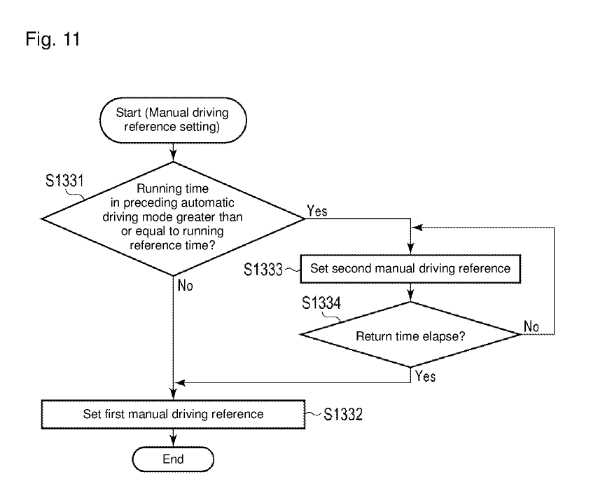

FIG. 11 is a flowchart illustrating a third reference setting example of the manual driving mode by the reference setting unit 2318a. The reference setting unit 2318a sets the first or second manual driving reference in the second period included in the execution period of the manual driving mode according to a running time of the vehicle in the first period included in the execution period of the automatic driving mode.

For example, the information acquisition unit 2317 acquires the running time. When the running time is shorter than a running reference time (NO in step S1331), the reference setting unit 2318a sets the first manual driving reference (step S1332). When the running time is greater than or equal to the running reference time (YES in step S1331), the reference setting unit 2318a sets the second manual driving reference (step S1333). The reference setting unit 2318a maintains the setting of the second manual driving reference (step S1333) until the return time (second period) elapses (NO in step S1334), and the reference setting unit 2318a changes the second manual driving reference to the first manual driving reference (step S1332) when the return time elapses (YES in step S1334). That is, the reference setting unit 2318a sets the first manual driving reference in the third period after the elapse of the second period.

The reference setting unit 2318a may set the first or second manual driving reference based on at least one of the running time, the running distance, and the road environment. For example, the reference setting unit 2318a may set the first manual driving reference in the case that the running distance is shorter than the running reference distance, and the reference setting unit 2318a may set the second manual driving reference when the running distance is greater than or equal to the running reference distance. The reference setting unit 2318a may set the first manual driving reference in the case that the road environment is the first road, and the reference setting unit 2318a may set the second manual driving reference in the case that the road environment is the second road. In the case that at least two conditions are combined, the reference setting unit 2318a may set the first manual driving reference when each condition satisfies the requirement for the setting of the first manual driving reference, and otherwise the reference setting unit 2318a may set the second manual driving reference.

The reference setting unit 2318a may set the first or second manual driving reference based on at least one of the driving concentration degree and the running information. In the case that both the conditions of the driving concentration degree and the running information are combined, the reference setting unit 2318a may set the first manual driving reference when each condition satisfies the requirement for the setting of the first manual driving reference, and otherwise the reference setting unit 2318a may set the second manual driving reference.

(Effect)

As described in detail above, in one embodiment of the present invention, the concentration degree determination device 2 can set the first reference for the manual driving mode that can be switched from the automatic driving mode or a second reference higher than the first reference based on at least one of the first driving concentration degree and the first running information during the execution of the automatic driving mode. That is, the risk degree determination reference can be set to the first or second reference based on the state during the execution of the automatic driving mode, and the second driving concentration degree during the execution of the manual driving mode can be determined based on the first or second reference that is variably set based on the state during the execution of the automatic driving mode. The information such as a warning can be output according to the determination result. Thus, depending on the state during the execution of the automatic driving mode, the driving safety is maintained by outputting information such as a proper warning during the execution of the manual driving mode. Depending on the state during the execution of the automatic driving mode, the comfort is maintained by outputting information such as a proper warning during the execution of the manual driving mode.

For example, in the case that the first driving concentration degree during the execution of the automatic driving mode is greater than or equal to the automatic driving reference, the risk degree is determined to be relatively low in the manual driving mode switched from the automatic driving mode, so that the first manual driving reference that is a standard for the safety can be set in the manual driving mode. In the case that the first driving concentration degree during the execution of the automatic driving mode is lower than the automatic driving reference, the risk degree is determined to be high in the manual driving mode switched from the automatic driving mode, so that the second manual driving reference that is strict for the safety can be set in the manual driving mode.

For example, in the case where the driver is awake during the execution of the automatic driving mode, the driver maintains the wakefulness state, and the risk degree is determined to be relatively low in the manual driving mode switched from the automatic driving mode, so that the first manual driving reference that is a standard for the safety can be set in the manual driving mode. In the case where the driver is asleep during the execution of the automatic driving mode, it takes time for the driver to return to the wakefulness state, so that the risk degree is determined to be high in the manual driving mode switched from the automatic driving mode, and the second manual driving reference that is strict for the safety can be set in the manual driving mode.

For example, in the case that the sleep time during the execution of the automatic driving mode is shorter than the sleep reference time, the driver is expected to return immediately to the wakefulness state, and the risk degree is determined to be relatively low in the manual driving mode switched from the automatic driving mode, so that the first manual driving reference that is a standard for the safety can be set in the manual driving mode. In the case that the sleep time during the execution of the automatic driving mode is greater than or equal to the sleep reference time, it takes time for the driver to return to the wakefulness state, so that the risk degree is determined to be high in the manual driving mode switched from the automatic driving mode, and the second manual driving reference that is strict for the safety can be set in the manual driving mode.

For example, in the case that the running time or the running distance during the execution of the automatic driving mode is shorter than the running reference time or the running reference distance, a fatigue degree of the driver is expected to be low, and the risk degree is determined to be relatively low in the manual driving mode switched from the automatic driving mode, so that the first manual driving reference that is a standard for the safety can be set in the manual driving mode. In the case that the running time or the running distance during the execution of the automatic driving mode is greater than or equal to the running reference time or the running reference distance, the fatigue degree of the driver is expected to be high, and the risk degree is determined to be high in the manual driving mode switched from the automatic driving mode, so that the second manual driving reference that is strict for the safety can be set in the manual driving mode.

For example, in the case that the road environment during the execution of the automatic driving mode is the first road, the fatigue degree of the driver is expected to be low, and the risk degree is determined to be relatively low in the manual driving mode switched from the automatic driving mode, so that the first manual driving reference that is a standard for the safety can be set in the manual driving mode. In the case that the road environment during the execution of the automatic driving mode is the second road, the fatigue degree of the driver is expected to be high, and the risk degree is determined to be high in the manual driving mode switched from the automatic driving mode, so that the second manual driving reference that is strict for the safety can be set in the manual driving mode.

For example, when the second period (given period) elapses after the second manual driving reference is set in the manual driving mode switched from the automatic driving mode, the second manual driving reference can be changed to the first manual driving reference in the manual driving mode. The driver is expected to be awakened even if the driver feels sleepy in the given period, and an excessive warning can be reduced to maintain the comfort by changing the strict second manual driving reference to the standard first manual driving reference.

For example, the second driving concentration degree during the execution of the manual driving mode switched from the automatic driving mode is monitored, and the second manual driving reference can be changed to the first manual driving reference in the case that at least a given time elapses while the second driving concentration degree is greater than or equal to the second manual driving reference. Consequently, the strict second manual driving reference can quickly be changed to the standard first manual driving reference in the case that the driving concentration degree is quickly increased in the manual driving mode, and the strict second manual driving reference is continuously set in the case that the driving concentration degree is low over a long period of time in the manual driving mode. This enables the excessive warning to be reduced to achieve compatibility between maintenance of the comfort and maintenance of the safety.

For example, a part of the automatic driving mode M11 can be set to the first period by adopting the first period of the first example in FIG. 4. For example, in the case that the execution period of the automatic driving mode M11 is extremely long, the restricted first period can be set, and the first or second manual driving reference can be set based on the first driving concentration degree corresponding to the restricted first period.

All the continuous execution periods of the automatic driving mode M11 can be set to the first period by adopting the first period of the second example in FIG. 5. Consequently, the first or second manual driving reference can be set based on the first driving concentration degree during the period from the start to the end of the automatic driving mode M11. The execution period of the plurality of discontinuous automatic driving modes can be set to the first period by adopting the first period of the third example in FIG. 6. Consequently, the first or second manual driving reference can be set based on the first driving concentration degree corresponding to the period of the plurality of discontinuous automatic driving modes.

### Other Embodiments

Other embodiments will be described below. In the embodiment, the proper use of the two references of the first and second manual driving references is described. Alternatively, at least three different references may properly be used. For example, the first manual driving reference is set in the case that one (first predetermined number) among a plurality of first driving concentration degrees corresponding to the plurality of indices obtained during the execution of the automatic driving mode does not satisfy the automatic driving reference. For example, the second manual driving reference is set in the case that three (a second predetermined number larger than the first predetermined number) among the plurality of first driving concentration degrees do not satisfy the automatic driving reference. A third manual driving reference higher than (stricter than) the second manual driving reference may be set in the case that five (a third predetermined number larger than the second predetermined number) among the plurality of first driving concentration degrees do not satisfy the automatic driving reference Alternatively, at least three manual driving references may properly be used based on a result of combining one or the plurality of first driving concentration degrees and the running information during the execution of the automatic driving mode.

In the embodiment, the first or second manual driving reference for the manual driving mode switched from the manual driving mode is set according to the determination as to whether the driver is asleep during the execution of the manual driving mode. Alternatively, the first or second manual driving reference may be set according to an index other than the determination as to whether the driver is asleep. For example, a determination as to whether the driver reads a book or plays a video game may be used instead of the determination as to whether the driver is asleep. Immediately after a person immerses himself or herself in reading or game play, sometimes the concentration is lowered, and the risk degree is increased. For example, the first automatic driving reference is set in the case where the driver does not read a book or play a video game, and the second automatic driving reference is set in the case where the driver reads a book or plays a video game.

In the embodiment, the concentration degree determination device 2 detects the state of the driver using the driver image data captured by the driver camera 15 as monitoring data, and estimates the driving concentration degree. However, the monitoring data is not limited to the driver image data. For example, the monitoring data may be biological data obtained by a biosensor that monitors the driver of the vehicle 1. For example, the biosensor is a pulse wave sensor or a heart beat sensor. The biosensor is not limited to the pulse wave sensor or the heart beat sensor as long as biosensor can monitor the driver. The biosensor may be a contact type sensor or a non-contact type sensor. The concentration degree determination device 2 can detect the state of the driver from the biological data. For example, the state of the driver detected from the biological data is an index such as a pulse wave or a heart beat. For example, the monitoring data may be data obtained by a sensor that is installed in the steering wheel 5 to measure strength of the driver who grasps the steering wheel 5.

In short, the present invention is not limited to the above embodiment, and constituent elements can be modified and embodied in the implementation stage without departing from the gist thereof. Various inventions can be made by appropriately combining a plurality of constituent elements disclosed in the above embodiment. For example, some constituent elements may be deleted from all the constituent elements illustrated in the embodiment. Constituent elements over different embodiments may appropriately be combined.

The embodiment may be implemented by a storage medium such as a read only memory (ROM) that stores a program causing the processor 231 to execute processing of each unit included in the processor 231.

A part or all of the above embodiment may also be described as follows, but is not limited to the following.

### (Supplementary Note 1)

A concentration degree determination device including:

a processor configured to acquire a driving concentration degree of a driver of a vehicle,

to set a first reference or a second reference higher than the first reference for a manual driving mode that is switched from an automatic driving mode based on at least one of a first driving concentration degree and running information during execution of the automatic driving mode,

to compare a second driving concentration degree during execution of the manual driving mode to the first or second reference, and

to output information according to a comparison result; and

a memory that stores an instruction to operate the processor.

### (Supplementary Note 2)

A concentration degree determination method including:

an information acquisition step of acquiring a driving concentration degree of a driver of a vehicle using at least one processor;

a reference setting step of setting a first reference or a second reference higher than the first reference for a manual driving mode that is switched from an automatic driving mode based on at least one of a first driving concentration degree and running information during execution of the automatic driving mode using the at least one processor;

a reference comparison step of comparing a second driving concentration degree during execution of the manual driving mode to the first or second reference using at least the one processor; and

an information output step of outputting information according to a comparison result using the at least one processor.

1. A concentration degree determination device comprising: a processor configured with a program to perform operations comprising:
operation as an information acquisition unit configured to acquire a driving concentration degree of a driver of a vehicle;
operation as a reference setting unit configured to set a first reference or a second reference higher than the first reference for a manual driving mode that is switched from an automatic driving mode based on at least one of a first driving concentration degree and running information during execution of the automatic driving mode in a first period comprising a period immediately before the automatic driving mode is switched to the manual driving mode;
operation as a reference comparator configured to compare a second driving concentration degree during execution of the manual driving mode to the first or second reference; and
operation as an information output unit configured to output information according to a comparison result.

2. The concentration degree determination device according to claim 1, wherein the processor is configured with the program perform operations such that operation as the reference comparator is further configured to set the first or second reference based on a sleep state of the driver comprised in an index of the first driving concentration degree.

3. The concentration degree determination device according to claim 1, wherein the processor is configured with the program perform operations such that operation as the reference comparator is further configured to set the first or second reference based on a sleep time of the driver comprised in an index of the first driving concentration degree.

4. The concentration degree determination device according to claim 1, wherein the processor is configured with the program perform operations such that operation as the reference setting unit is further configured to set the first or second reference based on at least one of a running time, a running distance, and a road environment that are comprised in the running information.

5. (canceled)

6. The concentration degree determination device according to claim 1, wherein the processor is configured with the program perform operations such that operation as the reference setting unit is further configured to set the first or second reference in a second period comprising a period immediately after the automatic driving mode is switched to the manual driving mode based on at least one of the first driving concentration degree and the running information.

7. The concentration degree determination device according to claim 6, wherein the processor is configured with the program perform operations such that operation as the reference setting unit is further configured to set the first reference in the manual driving mode after the second period elapses.

8. The concentration degree determination device according to claim 6, wherein the processor is configured with the program perform operations such that operation as the reference setting unit is further configured to set the first or second reference in the manual driving mode after the second period elapses based on the second driving concentration degree in the second period.

9. A concentration degree determination method comprising:
acquiring a driving concentration degree of a driver of a vehicle;
setting a first reference or a second reference higher than the first reference for a manual driving mode that is switched from an automatic driving mode based on at least one of a first driving concentration degree and running information during execution of the automatic driving mode in a first period comprising a period immediately before the automatic driving mode is switched to the manual driving mode;
comparing a second driving concentration degree during execution of the manual driving mode to the first or second reference; and
outputting information according to a comparison result.

10. A non-transitory computer-readable storage medium storing a program for determining concentration degree, the program, which when read and executed, causes a computer to perform the operations comprised in the concentration degree determination device according to claim 1.

11. The concentration degree determination device according to claim 2, wherein the processor is configured with the program perform operations such that operation as the reference comparator is further configured to set the first or second reference based on a sleep time of the driver comprised in the index of the first driving concentration degree.

12. The concentration degree determination device according to claim 2, wherein the processor is configured with the program perform operations such that operation as the reference setting unit is further configured to set the first or second reference based on at least one of a running time, a running distance, and a road environment that are comprised in the running information.

13. The concentration degree determination device according to claim 3, wherein the processor is configured with the program perform operations such that operation as the reference setting unit is further configured to set the first or second reference based on at least one of a running time, a running distance, and a road environment that are comprised in the running information.

14. The concentration degree determination device according to claim 2, wherein the processor is configured with the program perform operations such that operation as the reference setting unit is further configured to set the first or second reference in a second period comprising a period immediately after the automatic driving mode is switched to the manual driving mode based on at least one of the first driving concentration degree and the running information.

15. The concentration degree determination device according to claim 14, wherein the processor is configured with the program perform operations such that operation as the reference setting unit is further configured to set the first reference in the manual driving mode after the second period elapses.

16. The concentration degree determination device according to claim 14, wherein the processor is configured with the program perform operations such that operation as the reference setting unit is further configured to set the first or second reference in the manual driving mode after the second period elapses based on the second driving concentration degree in the second period.

17. The concentration degree determination device according to claim 3, wherein the processor is configured with the program perform operations such that operation as the reference setting unit is further configured to set the first or second reference in a second period comprising a period immediately after the automatic driving mode is switched to the manual driving mode based on at least one of the first driving concentration degree and the running information.

18. The concentration degree determination device according to claim 17, wherein the processor is configured with the program perform operations such that operation as the reference setting unit is further configured to set the first reference in the manual driving mode after the second period elapses.

19. The concentration degree determination device according to claim 17, wherein the processor is configured with the program perform operations such that operation as the reference setting unit is further configured to set the first or second reference in the manual driving mode after the second period elapses based on the second driving concentration degree in the second period.
